(19) United States
(12) Patent Application Publication
(10) Pub. No.: US 2021/0038117 A1
(43) Pub. Date: Feb. 11, 2021

(54) SMART CONTINUOUS POSITIVE AIRWAY PRESSURE SYSTEM AND METHOD

(71) Applicant: Bijan Sadrnoori, North Andover, MA (US)

(72) Inventor: Bijan Sadrnoori, North Andover, MA (US)

(21) Appl. No.: 16/832,145

(22) Filed: Mar. 27, 2020

Related U.S. Application Data
(60) Provisional application No. 62/883,444, filed on Aug. 6, 2019.

(51) Int. Cl. A61B 5/08; G06T 19/00; G06T 7/00; A61M 16/06

(52) U.S. Cl. CPC A61B 5/0826; G06T 19/006; G06T 7/0012; A61M 16/06; A61M 2205/3331; G06T 2207/30004; G06T 2207/20081; G06T 2210/41; G06T 2207/30201

(57) ABSTRACT

A system for determining continuous positive airway pressure (CPAP) and interface type for a user which includes one or more processors, one or more computer readable media, and one or more modules maintained on the one or more computer readable media that, when executed by the one or more processors, cause the one or more processors to perform operations including receiving an initial CPAP pressure setting for a user, monitoring a residual apnea hypopnea of the user, and determining an optimal CPAP pressure setting for the user based on the residual apnea hypopnea of the user.

### FIELD OF INVENTION

The present general inventive concept relates to a method of operating a continuous positive airway pressure (CPAP) system, and more particularly, to a system and method for determining and setting an effective CPAP pressure and interface type selected from a full face mask (FFM), nasal mask, and/or nasal pillow, a hybrid, or the like.

### DESCRIPTION OF RELATED ART

Sleep apnea affects over a billion people nationwide. As a result, continuous positive airway pressure (CPAP) systems and methods have been developed to treat apnea by delivering pressurized air or other breathable gas to the entrance of a patient's airways at a pressure elevated above atmospheric pressure.

Conventional CPAP systems and methods remain to be the preferred treatment for most patients with sleep apnea. However, the effectiveness of these current CPAP systems and methods are hindered by poor patient compliance attributable to poor mask fitting (i.e., interface) that causes leakage and arousal from sleep, and over or under pressurization of the pressure settings.

Therefore, what is desired is a continuous positive airway pressure (CPAP) system and method that determines the best pressure settings and interface type to maximize effectiveness of the sleep apnea treatment. In addition, what is desired is a CPAP system and method that reduces patient compliance issues and, as a result, reduces patient telephone calls to home healthcare providers and doctors.

### BRIEF SUMMARY OF THE INVENTION

The present general inventive concept provides a continuous positive airway pressure (CPAP) system.

The present general inventive concept also provides a method of using a continuous positive airway pressure (CPAP) system to determine the best pressure settings and CPAP mask interface type to maximize effectiveness of the sleep apnea treatment.

The present general inventive concept also provides a CPAP system and method that reduces patient compliance issues and, as a result, reduces patient telephone calls to home healthcare providers and doctors.

The present general inventive concept also relates a method and system for automatically determining and setting an effective CPAP pressure setting and facial interface type based on a captured 3D image of the patient's face.

Embodiments of the present general inventive concept provide a system for determining continuous positive airway pressure (CPAP) and CPAP mask interface type for a user including one or more processors, one or more computer readable media, and one or more modules maintained on the one or more computer readable media that, when executed by the one or more processors, cause the one or more processors to perform operations including receiving an initial CPAP pressure setting for the user, monitoring a residual apnea hypopnea of the user, and determining an optimal CPAP pressure setting for the user based on the residual apnea hypopnea of the user.

The CPAP system may include an adjustment module configured to adjust the initial CPAP pressure setting when the residual apnea hypopnea is greater than a predetermined value.

The CPAP system may include an image capturing module configured to capture an image of the user's face.

The captured image may include one of two-dimensional images and three-dimensional virtual reality images of the user's face in a supine position.

The CPAP system may include an overlaying module configured to overlay an image of an interface over the captured three-dimensional image of the user's face.

The CPAP system may include an optimal interface-type module configured to determine an optimate interface-type selected from a group consisting of a full mask, a nasal mask, a nasal pillow, and a hybrid based on the overlaid image of the interface over the captured three-dimensional image of the users face.

The CPAP system may include a sensor module configured to determine a high leak with a normal residual apnea hypopnea condition, a high leak with a high residual apnea hypopnea condition, and a high residual apnea hypopnea condition.

It is briefly noted that upon a reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention. As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The objects features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTIONS OF THE DRAWINGS

These and/or other aspects of the present general inventive concept will become apparent and more readily appreciated from the following description of the embodiments, taken in conjunction with the accompanying drawings of which:

FIG. 1 illustrates a schematic view of a continuous positive airway pressure (CPAP) system according to the present general inventive concept;

FIG. 2 illustrates a schematic view of various CPAP mask interface types;

FIG. 3 is a flowchart of a method of using the CPAP system according to an example embodiment of the present inventive concept;

FIG. 4 is a flowchart of a method of using the CPAP system according to another example embodiment of the present inventive concept;

FIG. 5 is a flowchart of a method of using the CPAP system according to another example embodiment of the present inventive concept;

FIG. 6 is a flowchart of a method of using the CPAP system according to another example embodiment of the present inventive concept; and

FIG. 7 is a flowchart of a method of using the CPAP system according to another example embodiment of the present inventive concept.

### DESCRIPTION OF INVENTION

Reference will now be made in detail to the embodiments of the present general inventive concept, examples of which are illustrated in the accompanying drawings, wherein like reference numerals refer to the like elements throughout. The embodiments are described below in order to explain the present general inventive concept by referring to the figures, however it will be understood that these embodiments are not intended to limit the present inventive concept to these embodiments alone. Instead, the present general inventive concept disclosed herein is intended to cover alternatives, modifications, and equivalents which may be included within the spirit and scope of the present disclosure as defined by the appended claims.

The present general inventive concept provides a continuous positive airway pressure (CPAP) system and method that automatically determines the best pressure settings and interface type to maximize effectiveness of the sleep apnea treatment.

The CPAP system according to the present general inventive concept is configured to resolve issues with patient compliance using conventional CPAP devices and to reduce frequency of mask and pressure changes, thereby reducing health care costs.

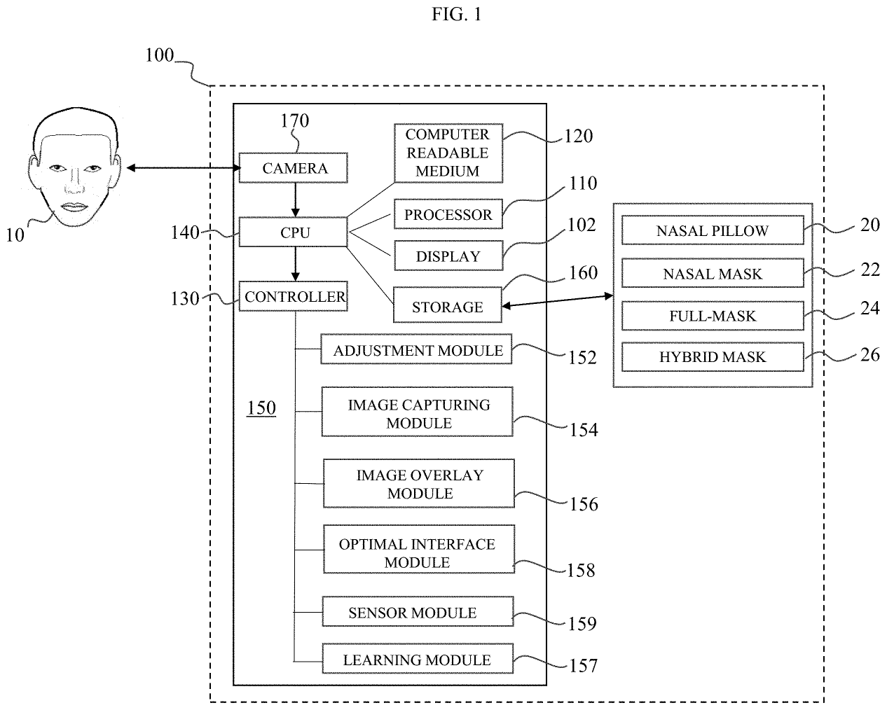

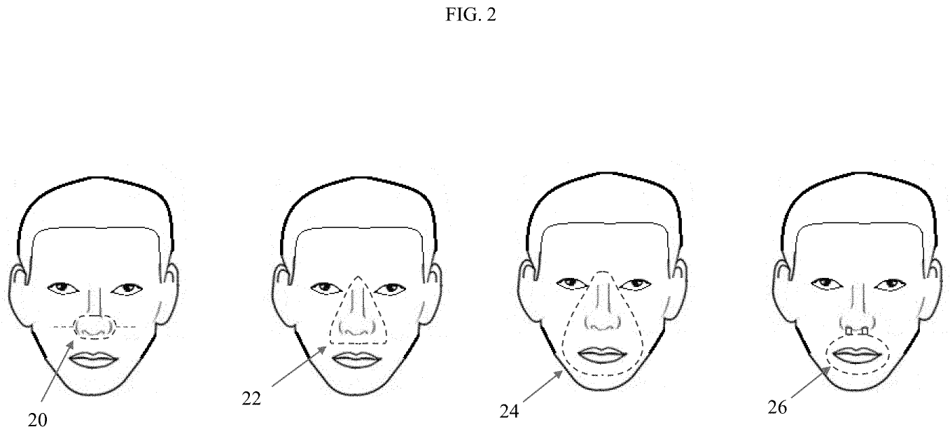

FIG. 1 illustrates a schematic view of a continuous positive airway pressure (CPAP) system 100 according to the present general inventive concept. FIG. 2 illustrates a schematic view of various CPAP mask interface types, including a nasal pillow 20, a nasal mask 22, a full-mask 24, and a hybrid mask 26.

Referring to FIG. 1, the continuous positive airway pressure (CPAP), designated generally as 100, is illustrated. In the present embodiment, the CPAP system 100 is designed and configured to automatically determine optimal pressure settings and CPAP mask interface types 20, 22, 24, 26 for each user 10. As a result, the CPAP system 100 according to the present general inventive concept is designed to reduce patient compliance issues and reduce user phone calls to health care providers, thereby reducing overall health care costs.

In the present embodiment, a CPAP system 100 automatically determines optimal pressure settings and mask interface type for a user 10 which includes one or more processors 110, one or more computer readable media 120, one or more controllers 130, one or more CPUs 140, and one or more modules 150 coupled to the one or more processors 110 and the one or more controllers 130. In response to a request by the one or more processors 110, the one or more controllers 140 cause the one or more processors 110 to perform operations defined within the computer readable media 120 and stored within a storage 160 including receiving an initial CPAP pressure setting for the user 10, monitoring a residual apnea hypopnea of the user 10, and then determining an optimal CPAP pressure setting and mask interface type for the user 10 based on the residual apnea hypopnea of the user 10. However, the present general inventive concept is not limited thereto.

In the present embodiment, the CPAP system 100 further includes an image capturing device (i.e., camera) 170 configured to capture an image of the user's 10 face.

In alternative embodiments, the CPAP system 100 further includes an adjustment module 152 configured to adjust the initial CPAP pressure setting when the residual apnea hypopnea is greater than a predetermined value.

In the present embodiment, the CPAP system 100 includes an image capturing module 154 configured to capture an image of the user's 10 face. The captured image includes one of two-dimensional images and three-dimensional virtual reality images. However, the present general inventive concept is not limited thereto.

In the present embodiment, the CPAP system 100 includes an overlaying module 156 configured to overlay an image of an interface over the captured three-dimensional image of the users face and an optimal interface-type module 158 configured to determine an optimum CPAP mask interface types selected from a group consisting of a nasal pillow 20, a nasal mask 22, a full mask 24, and a hybrid mask 26 stored in the storage 160 based on the overlaid image of the interface over the captured three-dimensional image of the users face.

In exemplary embodiment, the CPAP system 100 further includes a sensor module 159 configured to determine a high leak with a normal residual apnea hypopnea condition, a high leak with a high residual apnea hypopnea condition, and a high residual apnea hypopnea condition.

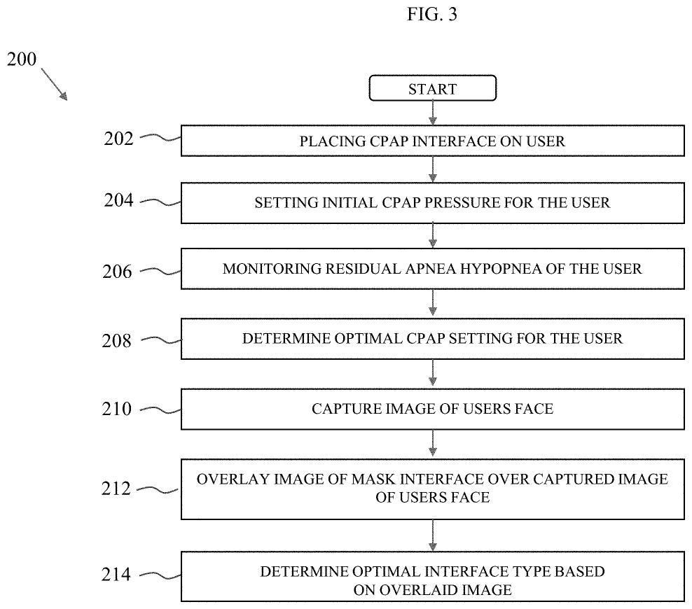

FIG. 3 is a flowchart of the CPAP system 100 and method 200 according to an example embodiment of the present inventive concept.

The continuous positive airway pressure (CPAP) system 100 according to present invention includes a custom designed instructions (i.e., algorithm) to perform operations defined within the computer readable media 120 and stored within a storage 160 and executed by the one or more CPU 140 of the CPAP system 100 to perform the following steps.

Referring to FIG. 3, the method 200 of using a continuous positive airway pressure (CPAP) system 100 begins at step 202 wherein a CPAP interface mask is placed over the user's 10 face. Next, at step 204, an initial CPAP pressure is set and used on the user 10. The initial CPAP pressure setting may be determined by a predicted formula and/or by a healthcare professional (e.g., doctor). However, the present general inventive concept is not limited thereto.

In the present embodiment, the CPAP system 100 is designed and configured to continuously and automatically to interact with the user 10 to adjust the initial CPAP pressure settings based on residual apnea hypopnea index (AHI) of the user 10. That is, if the user's AHI is normal the initial CPAP pressure setting is not changed and if the user's AHI is abnormal the initial CPAP pressure setting is changed by the CPAP system 100.

At step 206, a residual apnea hypopnea of the user 10 is continuously monitored using the sensor module 159 which is configured to monitor and detect the user's 10 residual apnea hypopnea condition.

At step 208, an adjustment module 152 of the CPAP system 100 is configured to determine an optimal CPAP pressure setting for the user 10 by automatically adjusting the initial CPAP pressure setting based on the user's 10 residual apnea hypopnea condition.

In alternative embodiments, the CPAP system 100 may then request whether or not the user 10 desires to change the initial CPAP pressure setting. If the user desires to change the initial CPAP pressure setting, a new CPAP pressure setting will then appear on a display 102 of the CPAP system 100. The user 10 may also select a previous CPAP pressure setting, if desired.

When the CPAP system 100 does not determine an optimal CPAP pressure setting, a red light will be illuminated on the display 102 of the CPAP system 100 indicating that one or more of the following is occurring with the user (1) central apneas; (2) periodic breathing; (3) ataxic breathing or (4) Biot's breathing.

When a large leak is detected, the CPAP system 100 requests the user 10 to select and use a different CPAP mask interface type 20, 22, 24, 26. If the user 10 agrees to select and use a different CPAP mask interface type 20, 22, 24, 26, the CPAP system 100 captures an image of the user's 10 face and nostrils in a supine position.

In the present embodiment, at step 210, the image capturing module 154 of the CPAP system 100 captures one of a two-dimensional or a three-dimensional virtual reality image of the user's 10 face and nostrils in a supine position.

In step 212, the overlaying module 156 of the CPAP system 100 overlays an image of a CPAP interface mask type 20, 22, 24, 26 over the captured three-dimensional virtual reality image of the user's 10 face.

Next, in step 214, the optimal interface-type module 158 of the CPAP system 100 determines an optimal CPAP mask interface type selected from a group consisting of a nasal pillow 20, a nasal mask 22, a full mask 24, and a hybrid mask 26 stored in the storage 160 based on the overlaid image of the CPAP interface mask type over the captured three-dimensional virtual reality image of the user's 10 face. The CPAP system 100 then overlays the type of interface (e.g., full face mask, nasal mask, nasal pillow, a hybrid, or the like) over the captured 3D virtual reality image of the user 10 in order to select the optimally fitted (i.e., best) CPAP mask interface type for the user 10 (e.g., patient). However, the present general inventive concept is not limited thereto.

In alternative embodiments, the CPAP system 100 further includes a CPAP learning module 157 that is configured to (1) determine whether there is a high leak with normal AHI; (2) determine whether there is a high leak with high AHI; (3) determine whether there is a high AHI (e.g., AHI=>10); (4) determine whether the pressure setting is greater than the reported downloaded pressure; and (5) illuminate a red light if proper CPAP pressure setting cannot be achieved.

According to the present general inventive concept, the CPAP system receives an initial pressure setting that may be set by a predicted formula and/or remotely by a medical doctor. Then, the CPAP system interacts with a patient and based on residual AHI, the CPAP system automatically determines that no change in pressure settings is required, when the patient's AHI is normal, or determines that a change in the pressure setting is required, when the patient's AHI is abnormal.

Next, the CPAP system will then request the patient if he or she wishes to change the initial CPAP pressure settings. If the patient agrees to change the initial pressure settings, the CPAP system captures a virtual reality image (e.g., two-dimensional or three-dimensional) of the patient's face.

The CPAP system then superimposes an image of a CPAP mask interface (e.g., full face mask, nasal mask, nasal pillow, a hybrid, or the like) over the captured virtual reality image of the patient's mouth and nostrils in a supine position to determine which CPAP mask interface is best suited for the particular patient based on his or her face dimensions and shape.

The CPAP system according to the present invention then determines which CPAP mask interface is suited for the patient and the healthcare provider orders the desired CPAP mask interface for the patient. However, the present general inventive concept is not limited thereto.

In addition, the CPAP system further includes sensors to detect and record timing when the CPAP system is on and off, and timing when the patient is in bed (or asleep) versus rise time (awake). The CPAP system may then calculate a nominator versus denominator. If this does not match, the CPAP system then determines that the patient has not been using the CPAP system from the beginning to end of sleep, which in turn may cause patient sleepiness, in spite of satisfactory downloading. However, the present general inventive concept is not limited thereto.

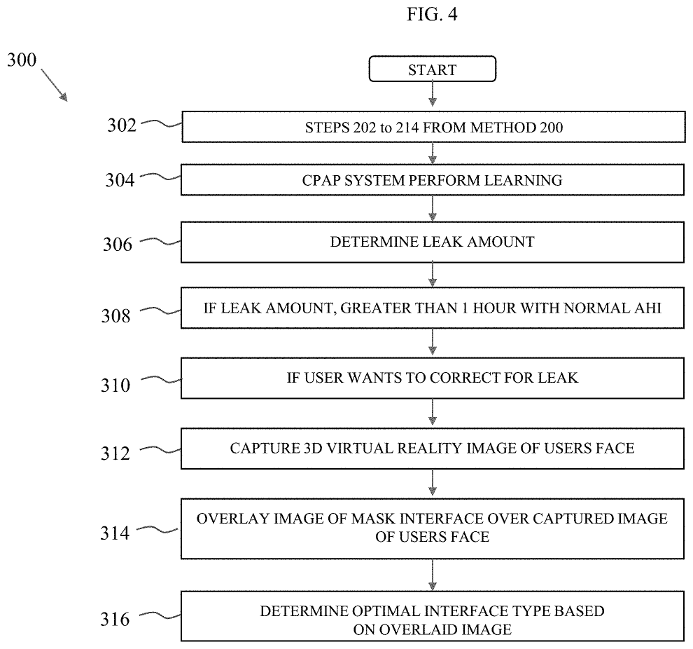

FIG. 4 is a flowchart of a method 300 of using the CPAP system 100 according to another example embodiment of the present inventive concept.

Referring to FIG. 4, the method 300 of using a continuous positive airway pressure (CPAP) system 100 begins at step 302 which includes the steps provided in method 200 as disclosed herein.

Next, at step 304, the CPAP system 100 performs a learning procedure.

At step 306, the CPAP system 100 determines a leak amount of the CPAP interface on the user.

At step 308, if the CPAP system 100 determines that the leak amount is greater than 1 hour with normal AHI, the system 100 will then output to a display screen a request for the user to indicate whether or not a correction is required. (Step 310). If the user requests to correct for the leak, the system 100 will then capture a 3D virtual reality image of the user's face. (Step 312).

At step 314, the system 100 will then overlay an image of mask interface types 20, 22, 24, and 26 (see FIG. 2) over the captured image of the user's face.

At step 316, the system 100 will then automatically select an optimal interface type (20, 22, 24, and 26) based on the overlaid image.

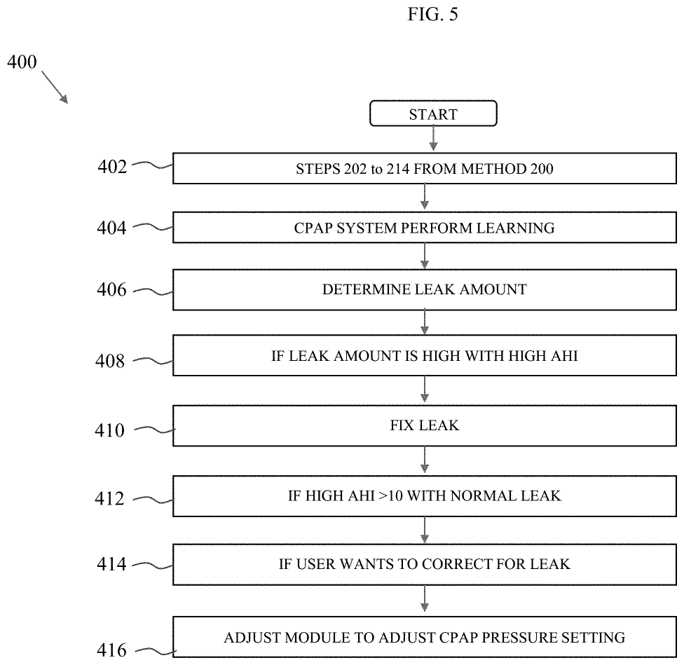

FIG. 5 is a flowchart of a method 400 of using the CPAP system 100 according to another example embodiment of the present inventive concept.

Referring to FIG. 5, the method 400 of using a continuous positive airway pressure (CPAP) system 100 begins at step 402 which includes the steps provided in method 200 as disclosed herein.

Next, at step 404, the CPAP system 100 performs a learning procedure.

At step 406, the CPAP system 100 determines a leak amount of the CPAP interface on the user.

At step 408, if the CPAP system 100 determines that the leak amount is high with a high AHI, the system 100 fixes the leak. (Step 410)

At step 412, if the CPAP system 100 determines that the leak amount is normal with a high AHI>10, the user is then requested to fix the leak.

At step 416, the CPAP system 100 uses adjust module to automatically adjust the user's CPAP pressure setting.

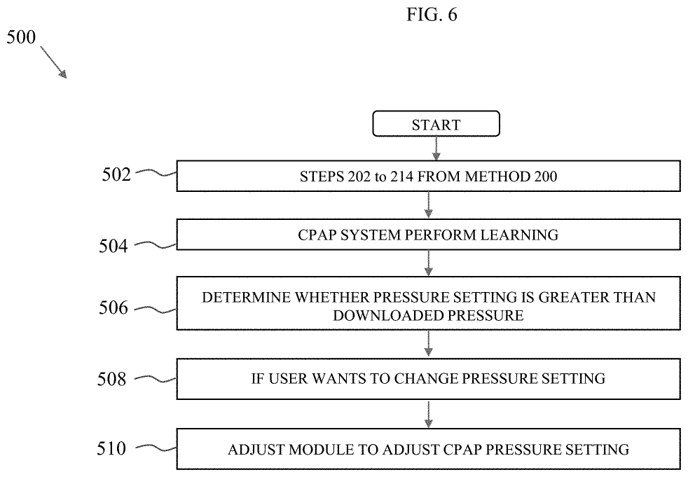

FIG. 6 is a flowchart of a method 500 of using the CPAP system 100 according to another example embodiment of the present inventive concept

Referring to FIG. 6, the method 500 of using a continuous positive airway pressure (CPAP) system 100 begins at step 502 which includes the steps provided in method 200 as disclosed herein.

Next, at step 504, the CPAP system 100 performs a learning procedure.

At step 506, the CPAP system 100 determines whether the CPAP pressure setting is greater than a downloaded pressure.

At step 508, if the user wants to change the CPAP pressure setting, the adjust module 152 of the CPAP system 100 adjusts the CPAP pressure setting.

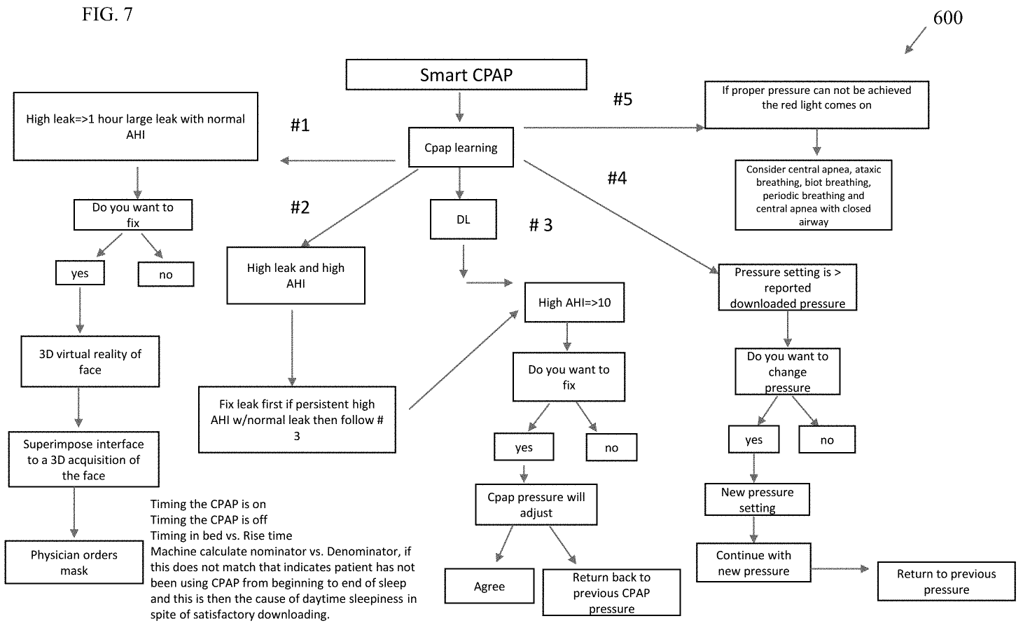

FIG. 7 is a flowchart of a method of using the CPAP system according to another example embodiment of the present inventive concept which illustrates a complete integrated system.

Although a few exemplary embodiments of the present general inventive concept have been illustrated and described, it will be appreciated by those skilled in the art that changes may be made in these exemplary embodiments without departing from the principles and spirit of the general inventive concept, the scope of which is defined in the appended claims and their equivalents.

What is claimed is:

1. A method for determining continuous positive airway pressure (CPAP) and interface type for a user, the method comprising:
under control of one or more processors configured with executable instructions,
receiving an initial CPAP pressure setting for a user;
monitoring a residual apnea hypopnea of the user; and
determining an optimal CPAP pressure setting for the user based on the residual apnea hypopnea of the user.

2. The method of claim 1, further comprising adjusting the initial CPAP pressure setting when the residual apnea hypopnea is greater than a predetermined value.

3. The method of claim 1, further comprising capturing an image of the users face.

4. The method of claim 3, wherein the image includes one of two-dimensional images and three-dimensional virtual reality images.

5. The method of claim 4, further comprising overlaying an image of an interface over the captured three-dimensional image of the users face.

6. The method of claim 5, further comprising determining an optimal interface type selected from a group consisting of a full mask, a nasal mask, a nasal pillow, and a hybrid based on the overlaying of the image of the interface over the captured three-dimensional image of the users face.

7. The method of claim 1, further comprising determining a high leak with a normal residual apnea hypopnea condition, a high leak with a high residual apnea hypopnea condition, and a high residual apnea hypopnea condition.

8. A system for determining continuous positive airway pressure (CPAP) and interface type for a user comprising:
one or more processors;
one or more computer readable media; and
one or more modules maintained on the one or more computer readable media that, when executed by the one or more processors, cause the one or more processors to perform operations including:
receiving an initial CPAP pressure setting for a user;
monitoring a residual apnea hypopnea of the user; and
determining an optimal CPAP pressure setting for the user based on the residual apnea hypopnea of the user.

9. The system of claim 8, further comprising an adjustment module configured to adjust the initial CPAP pressure setting when the residual apnea hypopnea is greater than a predetermined value.

10. The system of claim 8, further comprising an image capturing module configured to capture an image of the user's face.

11. The system of claim 10, wherein the captured image includes one of two-dimensional images and three-dimensional virtual reality images.

12. The system of claim 11, further comprising an overlaying module configured to overlay an image of an interface over the captured three-dimensional image of the users face.

13. The system of claim 12, further comprising an optimal interface-type module configured to determine an optimate interface-type selected from a group consisting of a full mask, a nasal mask, a nasal pillow, and a hybrid based on the overlaid image of the interface over the captured three-dimensional image of the users face.

14. The system of claim 13, further comprising a sensor module configured to determine a high leak with a normal residual apnea hypopnea condition, a high leak with a high residual apnea hypopnea condition, and a high residual apnea hypopnea condition.
